(19) United States
(12) Patent Application Publication
(10) Pub. No.: US 2021/0106714 A1
(43) Pub. Date: Apr. 15, 2021

(54) METHOD FOR VIRAL INACTIVATION BY CONTROLLED HYGROSCOPY

(71) Applicant: CENTRE SCIENTIFIQUE ET TECHNIQUE DU BATIMENT (CSTB), CHAMPS SUR MARNE (FR)

(72) Inventors: Thi Lan HA, EZANVILLE (FR); Sébastien RITOUX, NOISY LE GRAND (FR); Enric ROBINE, CONCHES SUR GONDOIRE (FR)

(21) Appl. No.: 16/956,772

(22) PCT Filed: Dec. 18, 2018
(86) PCT No.: PCT/EP2018/085450

§ 371 Date: Jun. 22, 2020

(30) Foreign Application Priority Data
Dec. 22, 2017 (FR) 1762986

(51) Int. Cl. A61L 9/14

(52) U.S. Cl. CPC A61L 9/14; A61L 2202/25; A61L 2209/16; A61L 2209/134; A61L 2209/132

(57) ABSTRACT

The present invention relates to a method for viral decontamination of a confined space, in particular of the air present in a confined space, based on application of a relative humidity of at least 50% created in said confined space, or incorporated at the level of a centralized air treatment system or an autonomous air purifier.

The present invention relates to the field of purification of the ambient air, notably in confined spaces, and aims to propose a new method dedicated more particularly to viral decontamination of the air.

There is a constant health need for controlling the quality of the ambient air, both in private premises and in public places, as 15000 liters of air passes through our respiratory tract every day. People in fact spend by far the greater part of their time indoors, where they are exposed to an ever increasing number of pollutants, and notably biological contaminants such as bacteria and viruses. Most office buildings, public transport systems, private cars and places for community life such as schools, child-care centers and cinemas for example have systems for ventilation, heating and air conditioning that are contaminated by such pollutants. This contamination of the ambient air is exacerbated, and is even more dangerous, in care establishments and other health facilities, human or animal, in which there is inevitably a larger number of sick individuals who are carriers of bacteria or viruses as well as a large number of weakened persons who are more likely to develop new health problems after being exposed to these pollutants.

A well-known method for removing these contaminants from the ambient air consists of using air purifiers. These air purifiers use various techniques such as exposing the contaminants to ultraviolet light, filtration of the contaminants, adsorption of the contaminants, the use of active oxygen or of hydroxyl ions, photoionization or else air purification by combustion, by plasma or by photocatalysis.

However, these techniques are not found to be completely satisfactory in terms of efficacy and cost.

For example, most air purifiers use an ultraviolet lamp that does not allow a sufficient exposure time of the pathogens in the air to obtain a satisfactory result.

Air purification by filtration is expensive, and even filter replacement must comply with standards that are so strict that use thereof by private individuals is limited thereby. Photoionization is not able to treat contaminants such as bacteria and viruses present in the air. Purification by combustion is a very slow process, which is only effective in very small spaces. Air purification by plasma is very expensive, this recent technology having been developed by the aerospace industry. Purification by photocatalysis requires the catalytic medium used (most often TiO₂) to be free from any impurities, such as dust. Moreover, purifiers using this technology are very noisy. Finally, air purification by adsorption is also very expensive, as it is a recent technology, controlled by a small group of manufacturers.

There is therefore a need for a new method capable of efficiently degrading viruses present in the air, at reasonable cost and in a reasonable time. There is also a need for a method by which individuals present in a confined space can have their exposure to respiratory viruses reduced.

Unexpectedly, the inventors have found that it is possible to inactivate the viral agents present in the air of a confined space by controlling the humidity level in this space so as to generate a relative humidity of at least 50%. As illustrated in the examples, the inventors were in fact able to decontaminate a room that was completely contaminated beforehand, in a controlled manner, with an infectious virus model of the Influenza virus, reducing its viral load completely.

Thus, according to its first aspect, the present invention relates to a method for viral decontamination of a confined space, in particular of the air present in a confined space, in which a relative humidity of at least 50% is created in said confined space, for a predetermined length of time.

According to certain embodiments, humidity is generated in an air treatment device, such as an air conditioning device or a centralized or autonomous air treatment system, prior to introduction of the humidified air into said confined space.

According to certain other embodiments, humidity is generated directly in said confined space, for example by water misting in said confined space, by means of any water misting device known by a person skilled in the art.

Preferably, a relative humidity of at least 60%, preferably at least 70%, preferably of 70%, is created in said confined space. Thus, according to these embodiments, the predetermined relative humidity is created in said confined space by water misting. This water misting is preferably carried out in the absence of antiviral active agents, and in particular in the absence of virucidal active agents.

According to certain embodiments, said confined space is selected from private or public spaces, in particular from a dwelling; a cold room; a waiting room; a doctor's office; a clinic; a hospital; an operating theater for human or veterinary medicine; a school; a child-care center; a station; an office; a meeting room; a movie theater; a means of transport such as a vehicle, notably refrigerated, a wagon and an airplane cabin or boat cabin; a container, a centralized air treatment system and an air purifier, notably an autonomous air purifier.

In particular, a temperature of at least 25° C., in particular of at least 30° C. and preferably of 30° C. may be generated in said confined space.

According to one embodiment, the relative humidity described above is created in said confined space for a length of time between 1 hour and 20 hours, preferably between 2 hours and 15 hours, in particular between 5 hours and 14 hours, and preferably between 6 hours and 14 hours.

A method according to the invention advantageously makes it possible to inactivate viruses present in said confined space, notably in the air of said confined space.

They are preferably human pathogenic viruses.

These viruses are preferably enveloped RNA viruses, in particular selected from the viruses belonging to the families Cystoviridae, Retroviridae, Reoviridae, Orthomyxoviridae and Paramyxoviridae. These viruses may more particularly be selected advantageously from the viruses of the genera cystovirus, lentivirus, epsilonretrovirus, alpharetrovirus, betaretrovirus, gammaretrovirus, deltaretrovirus, orthoreovirus, orbivirus, rotavirus, coltivirus, aquareovirus, cypovirus, fijivirus, phytoreovirus, oryzavirus, idnoreovirus, mycoreovirus, influenzavirus, is avirus, thogotovirus, avulavirus, henipavirus, morbilivirus, respirovirus, rubulavirus, pneumovirus and metapneumovirus. It may more particularly be viruses of the genus influenzavirus.

### DESCRIPTION OF THE FIGURES

FIG. 1 illustrates the variation of the concentration of particles with a size between 0.5 and 1.65 μm during experiments conducted in the enclosure as described in example 2 below.

Abscissa: particle collection time, in minutes. Ordinate: concentration of particles per liter.

FIG. 2 illustrates the lysis plaque reading of a Phi6 phage in the context of the measurement of infectious viruses described later on in the present text.

FIG. 3 illustrates the effect of variation of the relative humidity in the enclosure on the concentration of infectious viruses in the air in said enclosure. The measurements are illustrated according to the site of air sampling, namely either in the enclosure surroundings, or at the level of air extraction.

Abscissa: relative humidity (RH) 30%, 50% or 70%. Ordinate: concentration of infectious viruses in the air, in PFU/m³.

### CONFINED SPACE

The present invention relates to a method for viral decontamination of a confined space. In particular, said method allows decontamination of the air of said space.

“Confined space” means an interior space, as opposed to an exterior space. Consequently it is an enclosed or semi-enclosed space, notably having at least one door, which may be open or closed.

There is no limit to the volume of this space, provided that this space is enclosed or semi-enclosed. Thus, it may be a vehicle, a centralized air treatment system, an air purifier, notably an autonomous air purifier, as well as a larger space, such as for example a movie theater or concert hall, a gymnasium or a shopping mall.

As examples of confined spaces suitable for the invention, we may notably mention confined spaces selected from private or public spaces, in particular from a dwelling; a cold room; a waiting room; a doctor's office; a clinic; a hospital; an operating theater for human or veterinary medicine; a school; a child-care center; a station; an office; a gymnasium; a concert hall; a theater; a meeting room; a movie theater; a shopping mall; a means of transport such as a vehicle, notably refrigerated, a wagon, an airplane cabin or boat cabin; a container, a centralized air treatment system and an air purifier, notably an autonomous air purifier.

Creation of a Defined Relative Humidity

Numerous means for varying, and notably increasing, the relative humidity of a place are well known by a person skilled in the art.

The relative humidity of the air, also called air humidity or φ, corresponds to the ratio of the partial pressure of the water vapor contained in the air to the saturated vapor pressure at the same temperature.

The operation of adjusting the relative humidity of the air mainly consists of spraying an aqueous liquid, in particular water, into said air. This aqueous liquid, and in particular water, is advantageously sprayed into the confined space in question in gaseous form.

It is consequently the ratio of the content of water vapor in the air to its maximum capacity for containing it in given conditions.

The relative humidity of a space considered by the present invention can be measured using a hygrometer. This instrument is familiar to a person skilled in the art.

For example, the relative humidity of a space such as mentioned above may be measured using a hair hygrometer, a condensation hygrometer, a resistive hygrometer or an capacitive hygrometer.

The relative humidity of a space as described above may be increased by any means known by a person skilled in the art.

As an illustration, a humidifier may be used, such as for example a boiling humidifier, an ultrasonic humidifier, a mister, such as an ultrasonic mister or a nebulizer.

According to one embodiment, the method according to the invention is characterized in that the relative humidity is generated in an aforementioned confined space by water misting, preferably only by water misting.

Alternatively, the relative humidity of a space as described above may be decreased by any means known by a person skilled in the art.

As an illustration, a condensation dehumidifier or a humidity absorber may be used in this sense.

In a method according to the invention, the relative humidity of a confined space as described above is at least 50%.

According to a particular embodiment, this relative humidity is at least 60%, preferably at least 70% and preferably 70%.

As stated above, a method according to the invention is advantageously characterized in that the relative humidity is generated for a predetermined length of time, i.e. before applying the method.

According to one embodiment of the method according to the invention, the relative humidity described above is created in said confined space for a length of time between 10 minutes hour and 10 hours, preferably between 20 minutes and 8 hours, in particular between 30 minutes and 5 hours, and preferably between 45 hours and 3 hours.

According to one embodiment, the temperature created in the confined space as described above during a method according to the invention is at least 15° C., in particular at least 20° C., and is preferably 20° C.

Effect of the Method According to the Invention

A method according to the invention advantageously allows viral decontamination of a confined space as defined above, in particular of the air contained in this confined space.

“Viral decontamination” means the removal, partial or complete, of the viruses present in the air or on the surface of objects in question. This removal may consist of the destruction or the inactivation of said viruses.

“Inactivation of a virus” or “viral inactivation” means making a virus inactive or noninfectious. An inactive or noninfectious virus denotes a virus rendered ineffective, i.e. incapable of causing the disease that it causes or may cause in an individual when it is active.

Thus, according to a preferred embodiment, a method according to the invention makes it possible to inactivate viruses present in a confined space as defined above, notably in the air of said confined space.

Advantageously, a method for viral decontamination according to the invention is used in the absence of an antiviral active agent, in particular in the absence of a virucidal active agent.

According to one embodiment, the viruses more particularly targeted by a method according to the invention are the enveloped RNA viruses. They may in particular be viruses selected from the viruses belonging to the families Cystoviridae, Retroviridae, Reoviridae, Orthomyxoviridae and Paramyxoviridae.

More particularly, these viruses may be selected from the viruses of the genera cystovirus, lentivirus, epsilonretrovirus , alpharetrovirus, betaretrovirus, gammaretrovirus, deltaretrovirus, orthoreovirus, orbivirus, rotavirus, coltivirus, aquareovirus, cypovirus, fijivirus, phytoreovirus, oryzavirus, idnoreovirus, mycoreovirus, influenzavirus, is avirus, thogotovirus, avulavirus, henipavirus, morbilivirus, respirovirus, rubulavirus, pneumovirus and metapneumovirus.

These viruses may in particular be of the genus Influenza.

The expressions “between . . . and . . . ” and “from . . . to . . . ” are to be understood as inclusive, unless stated otherwise. Moreover, the expression “at least [value X]” means including the lower bound. Thus, the expression “at least 70%” means that the value of 70% is included.

### EXAMPLE

### Example 1

### Application of a Method of Decontamination According to the Invention on the Scale of a Realistic Environment

The virostatic potential of air humidification was assessed in realistic conditions. For this purpose, the methodology was based on carrying out tests within the “classroom” space, called the enclosure hereinafter, simulated in the experimental enclosure of the Centre Scientifique et Technique du Bâtiment (Building Scientific and Technical Center).

The evaluation procedure used, as well as the performance of this method, are described hereunder.

Phi6 phages, as well as their host strain Pseudomonas syringae, supplied by the bacterial virus reference center, Félix Hérelle, were used as described below after amplification thereof. This amplification was carried out according to the protocols elaborated by the IUCPQ, as described in Turgeon et al. (2014, Applied and Environmental Microbiology, Vol. 80: 4242-4250.

The Phi6 virus is an enveloped RNA virus, with a diameter of 85 nm. This virus is incubated in Trypto-Casein Soy Agar as culture medium, at a temperature of 25° C. and constitutes in particular a model of eukaryotic viruses for the Reoviridae (notably the rotaviruses), the Orthomyxoviridae (notably influenza), the Paramyxoviridae (in particular the measles virus) and the Retroviridae (notably HIV).

Evaluation Methodology

Controlled viral contamination was reproduced in the test space (enclosure) for assessing the performance of the method/decontamination procedure.

For these tests, suspensions of Phi 6 phages, of 3-5.10⁹ PFU/mL, were nebulized directly in the space for 5 minutes using a Collison 6 jets generator (BGI).

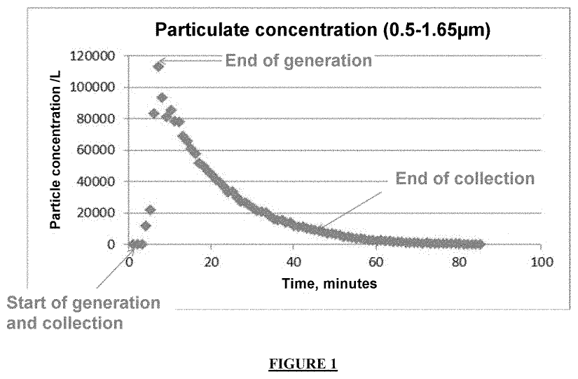

To evaluate the effect of the method of treatment employed on this viral load, the metrology used is based on aerosol sampling followed by analysis of the infectious phage titer. Thus, the phage aerosol was collected using two liquid medium samplers (BioSampler® sold by SKC INC.) placed in the surroundings and at the level of the air extraction duct. The BioSamplers® are started at the start of injection and operate for 45 minutes, this collection time making it possible to integrate most of the phage pollution generated, as shown by monitoring the concentration of the particles in the space during the experiments. The concentration of particles is monitored during the experiments using an optical counter (model 1.108, Grimm) placed in the space (see FIG. 1).

At the end of sampling, the infectious virus load (PFU/m³) is analyzed by any method known by a person skilled in the art, for example such as the method described by Trouwborst et al. (1974, J Gen Virol London, Vol. 24:155-165), or else by the method described in the examples.

Measurement of the Infectious Viruses

Measurement of the infectious titer is based on counting the lesions produced by phage infection of bacterial cultures. In the presence of the right host strain, the bacterium-phage mixture will cause, after incubation, confluent lysis of the bacterial culture.

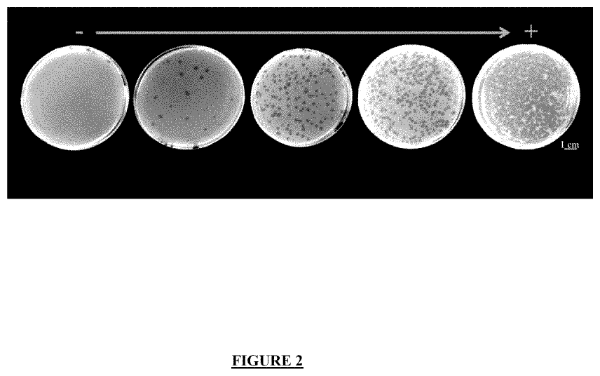

In practice, serial dilutions of the phage solutions to be titrated are seeded simultaneously with the cells of the host bacteria, in TSA soft agar medium. The cultures are then incubated at the appropriate temperatures until a lysis plaque is observed after about 24° C. A lysis plaque corresponds to a clear or even a translucent region on a dense bacterial culture. Counting the lysis plaques makes it possible to determine the infectious titer, expressed in PFU or plaque-forming unit per ml, from the following formula:

$$\frac{PFU}{mL} = \frac{PFU}{V \times D}$$

where D and V correspond to the phage dilution and the volume in mL deposited in the soft agar mixture, respectively. Only the cultures comprising between 15 and 300 lysis plaques were counted (NF EN ISO 7218/A1, 2013). FIG. 2 Error! Reference source not found. illustrates the levels of lysis plaques observable.

Results of Decontamination by Air Humidification According to the Invention

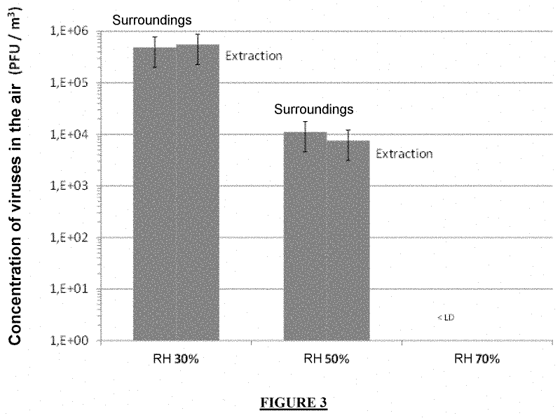

FIG. 3 presents the concentration of infectious viruses in the air according to the relative humidity employed in the enclosure.

We observe a decrease in infectious viral load with increase in relative humidity of the room, with, as a corollary, lowering of the level of exposure to the virus. A reduction of 2 logs is observed between 30 and 50% relative humidity, and an additional reduction of 4 logs is observed on passing from 50 to 70% relative humidity.

The results are statistically identical, whether sampling was effected in the surroundings or at the level of air extraction.

“In the surroundings” means sampling carried out at the height of the respiratory tract, at different points of the enclosure.

These results thus demonstrate the efficacy of a method according to the invention for inactivating viruses in suspension in the air of a confined space.

1. A method for viral decontamination of a confined space, in particular of the air present in a confined space, in which a relative humidity of at least 50% is created in said confined space, for a predetermined length of time.

2. The method as claimed in claim 1, in which a relative humidity of at least 60%, preferably at least 70%, preferably of 70%, is created in said confined space.

3. The method as claimed in claim 1, in which the predetermined relative humidity is created in said confined space by water misting, preferably in the absence of antiviral active agent, in particular in the absence of virucidal active agent.

4. The method as claimed in claim 1, in which said confined space is selected from private or public spaces, in particular from a dwelling; a cold room; a waiting room; a doctor's office; a clinic; a hospital; an operating theater for human or veterinary medicine; a school; a child-care center; a station; an office; a gymnasium; a concert hall; a theater; a meeting room; a movie theater; a shopping mall; a means of transport such as a vehicle, notably refrigerated, a wagon and an airplane cabin or boat cabin; a container, a centralized air treatment system and an air purifier, notably an autonomous air purifier.

5. The method as claimed in claim 1, in which a temperature of at least 15° C., in particular of at least 20° C. and preferably of 20° C. is created in said confined space.

6. The method as claimed in claim 1, in which the relative humidity described above is created in said confined space for a length of time between 10 minutes and 10 hours, preferably between 20 minutes and 8 hours, in particular between 30 minutes and 5 hours, and preferably between 6 hours and 14 hours.

7. The method as claimed in claim 1, for inactivating viruses present in said confined space, notably in the air of said confined space.

8. The method as claimed in claim 7, said viruses being enveloped RNA viruses, in particular selected from the viruses belonging to the families Cystoviridae, Retroviridae, Reoviridae, Orthomyxoviridae and Paramyxoviridae.

9. The method as claimed in claim 8, said virus being selected from the viruses of the genera cystovirus, lentivirus, epsilonretrovirus, alpharetrovirus, betaretrovirus, gammaretrovirus, deltaretrovirus, orthoreovirus, orbivirus, rotavirus, coltivirus, aquareovirus, cypovirus, fijivirus, phytoreovirus, oryzavirus, idnoreovirus, mycoreovirus, influenzavirus, isavirus, thogotovirus, avulavirus, henipavirus, morbilivirus, respirovirus, rubulavirus, pneumovirus and metapneumovirus, and is in particular a virus of the genus influenzavirus.
